SEQUENCE LISTING (2 sequences)

SEQ ID NO: 1 - protein, 345 aa, Varicella zoster
MGWSCIILFLVATATGVHSSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVHHHHHH

SEQ ID NO: 2 - protein, 339 aa, Varicella zoster
MGWSCIILFLVATATGVHSSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHV

(19) United States
(12) Patent Application Publication
(10) Pub. No.: US 2026/0367083 A1
(43) Pub. Date: Nov. 19, 2026

(54) Varicella Zoster Virus Lateral Flow Assay

(71) Applicant: VIRO RESEARCH, LLC, Snellville, GA (US)

(72) Inventors: Nicholas Vafai, Snellville, GA (US); Abbas Vafai, Snellville, GA (US)

(73) Assignee: VIRO RESEARCH, LLC, Snellville, GA (US)

(21) Appl. No.: 18/273,170

(22) PCT Filed: Jan. 20, 2022
(86) PCT No.: PCT/US2022/013126

§ 371 Date: Jul. 19, 2023

(51) Int. Cl. G01N 33/569; C07K 14/005; G01N 33/543

(52) U.S. Cl. CPC G01N 33/56994; C07K 14/005; G01N 33/54388; C12N 2710/16722; G01N 2333/04; G01N 2469/20

(57) ABSTRACT

Generally, the present disclosure is directed to an antibody detection lateral flow assay that is specific to antibodies binding a portion of the gE protein expressed by VZV. The assay can be based on visual labels and interpreted without the use of a reader to provide for quick and easy detection. Multiple colored labels can be used for evaluation, and these colored labels can be covalently attached to the portion of the gE protein and the resulting modified gE-label complex applied to a substrate (e.g., backing card) for use in a binding assay.

### SEQUENCE LISTING

The content of the electronic sequence listing (Sequence Listing from 064180-00020_ST25.txt; Size 8 KB; File Metadata Date of Creation: Nov. 19, 2025; Original Copy Date of Creation on or before May 5, 2022) submitted herewith, is herein incorporated by reference in its entirety.

### FIELD

The present application is directed to systems, methods, and apparatuses for antibody detection. More particularly, the present disclosure presents an example lateral flow assay for detection of antibodies in human serum/blood against Varicella-Zoster Virus (VZV) envelope glycoprotein E (gE) protein.

### BACKGROUND

Varicella-zoster virus (VZV) is the causative agent of childhood chickenpox (varicella) and shingles (zoster), two distinct clinical manifestations. Varicella is the outcome of the primary encounter (infection) with VZV, whereas zoster is the result of VZV reactivation which occurs predominantly in aging and immunosuppressed individuals, including cancer and AIDS patients.

There are 2.5 million estimated cases of chickenpox and 1.2 million cases of shingles per year in the United States, and it is expected that the number of shingles patients will increase as the population ages.

One of the most common complications of shingles includes postherpetic neuralgia which is characterized by intractable pain lasting for four weeks to several years after the onset of skin rash. Other complications of VZV reactivation (shingles) include encephalitis, pneumonitis and disseminated zoster.

VZV is a member of the alpha herpesvirus family, and VZV DNA encodes multiple glycoproteins that are commonly found in infected cells. These glycoproteins can produce immunogenic responses that elicit neutralizing antibody secretion.

Current VZV antibody detection systems utilize viral lysate as part of an ELISA capture system; however, these capture assays suffer several drawbacks including long read time, waste from excess reagents, and inability to work with typical patient samples such as whole blood. Still needed in the art are improved VZV antibody detection systems that can be designed for mass production via incorporation of expressed isolated peptide, and which can be used on patient samples as point-of-care. Further, such systems may be most beneficial if results can be validated within a short time frame (e.g., within 1 hour).

### SUMMARY

In one embodiment, the present application is directed to an antibody detection lateral flow assay that is specific to antibodies binding a portion of the gE protein expressed by VZV. In some aspects of this embodiment, the assay is based on visual labels and interpreted without the use of a reader to provide for quick and easy detection. In other embodiments, the assay is quantitative, and is designed for use with a reader system. In some embodiments, the assay is a sandwich assay, and in other embodiments, the assay is a competitive assay.

Multiple colored labels can be used for evaluation, including colloidal gold, colored latex, and cellulose nanobeads. These colored labels can be chemically or physically attached (for example, covalently bonded, ionically bonded, bonded by chemisorption or physisorption, bonded via functional linking groups or linker molecules, or bonded via hydrogen bonding, hydrophobic interaction, or Van Der Waals attraction), attached to the portion of the isolated gE protein and the resulting modified gE-label complex applied to a substrate (e.g., backing card) for use in a binding assay. Further, the gE protein (also referred to herein as an isolated gE peptide) can be isolated via an expression system, rather than a viral lysate, to produce a more uniform test composition that would likely display improved reproducibility relative to ELISA assays.

The assay involves testing a biological sample. The type of biological sample can be derived from any of several formats, such as whole blood venipuncture, finger stick sample, and/or isolated serum. According to various aspects of the disclosure, example assays can be designed to detect the IgG response, the IgM response or both. For instance, a sandwich type binding assay can include a first binding event where the blood sample is exposed to the gE-label complex under conditions such that antibodies present in the blood sample will bind to the portion of the gE protein. The binding assay can also include a second binding event where gE-label complex bound to an antibody migrates (e.g., through capillary action) to a region including the portion of the gE protein bound to a substrate. These two binding events lead to the formation of a complex including two portions of the gE protein—a first portion of the gE protein that is bound to the substrate and a second portion of the gE protein that is not bound and is linked to a label. Localized concentration of the complex in the area having the first portion of the gE protein bound to the assay leads to production of a signal that can be identified visually as one possible mechanism for indicating the presence of antibodies against VZV in the sample.

As an example for illustration, the antibody assay can be in the form of a test strip which includes a membrane (e.g., nitrocellulose), a conjugate pad, a sample pad, and a wick. The strip can be contained in a cassette, or include other casings to protect the assay and/or the stability of any material included in the assay.

In some embodiments, the assay may be in the form of a kit. The kit may include other reagents or materials for facilitating diagnosis such as a chase buffer to facilitate movement of the sample along the strip, a result indicator to facilitate interpretation of the assay, and/or a needle or stick to facilitate sample collection.

In another embodiment, the present disclosure is directed to a method for detecting the presence of VZV antibodies in a patient. The method can include applying a patient-derived biological sample to a sample pad, where capillary forces and the wick aid the movement of the sample through the sample pad and up the membrane. The sample fluid travels into a conjugate pad where labeled conjugate proteins interact with antibodies in the patient sample, if present. Then, the fluid travels over the test line on the membrane.

Generally, the test line includes the VZV gE protein that has already been striped and dried on the membrane. If positive, the patient's antibodies that have bound to the conjugates will bind to the VZV gE in a sandwich format. This will cause a visible change (e.g., a red/brown line) that can be seen by the eye. If negative, no line will be visible.

In some embodiments, the sample fluid may continue to run up the membrane, which may further include a control line. The control line can include antibodies or proteins already striped and dried on the membrane. The proteins or antibodies on the control line can be designed to bind to the labeled conjugate proteins, regardless of whether or not the labeled conjugate protein is bound to an antibody. For example, the labeled portion of the VZV gE protein can further include a covalent attachment to a control molecule that the proteins or antibodies on the control line can bind.

In some aspects of these embodiments, the sample is allowed to run on the test strip for a period of time, prior to making a determination of the result. For instance, wicking materials and/or peptide compositions may be adjusted to provide a faster determination of antibody presence in the patient sample. In this manner, certain implementations may provide an advantage in antibody detection at point of care (e.g., within one hour) which may reduce costs and lead to less stress.

In another embodiment, treatment methods for VZV are disclosed. In this embodiment, a patient obtains a biological sample, tests the sample on the test strip, and, if the sample tests positive for VZV infection, the patient can be treated with antiviral agents and/or other types of treatment for VZV infection. Whether or not a patient tests positive, the patient can also benefit from prophylaxis with a Shingles vaccine. Accordingly, in one aspect of this embodiment, the patient is encouraged to take a Shingles vaccine regardless of their testing status.

In still another embodiment, isolated peptides useful for carrying out the assays described herein are disclosed. The isolated peptides can include an amino acid sequence which comprises a portion of the gE protein.

(SEQ ID 1)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHVHHHHHH,
(SEQ ID 2)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHV,

Representative amino acid sequences include:

and analogues thereof.

In one embodiment, the isolated peptide is bound to a particle, such as a latex or a gold particle, and in some aspects of this embodiment, the isolated peptide is chemically or physically bound to the particle, for example, covalently bonded, ionically bonded, bonded by chemisorption or physisorption, bonded via functional linking groups or linker molecules, or bonded via hydrogen bonding, hydrophobic interaction, adsorption, absorption, or Van Der Waals attraction).

In another embodiment, the isolated peptide is chemically or physically bound to a substrate. In one embodiment, where the isolated peptide is bound to a substrate, the location where the isolated peptide is bound to the substrate is at the test line of an LFA. In another embodiment, the isolated peptide is also bound to the substrate at the control line of the LFA.

In another embodiment, the isolated peptide is bound to a particle, and also bound to a VZV antibody. In one aspect of this embodiment, the antibody in the “isolated peptide-particle-antibody conjugate” is also bound to an isolated peptide bound to a solid support. The conjugate (or complex) of the VZV antibody to a) the isolated peptide bound to a particle, and b) the isolated peptide bound to a solid support, is referred to as a “sandwich,” and is used in the sandwich assays described herein to indicate a user has tested positive for VZV infection.

### BRIEF DESCRIPTION OF THE DRAWINGS

Many aspects of the present disclosure will be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. It should be recognized that these implementations and embodiments are merely illustrative of the principles of the present disclosure.

FIG. 1 illustrates an example cassette for running a lateral flow assay in accordance aspects of example implementations of the present disclosure.

FIG. 2A illustrates a table of example results for a lateral flow assay using different plasma samples in accordance with aspects of example implementations of the present disclosure.

FIG. 2B illustrates a table of readings corresponding to the Axxin measured results in FIG. 2A.

FIG. 3 illustrates a bar graph comparing latex loading radios of 20:1 and 40:1 for different plasma samples.

FIG. 4 illustrates a line graph displaying treated (1% BSA, 0.5% Tergitol) vs. untreated Axxin measurements.

FIG. 5A illustrates a cartoon of an example sandwich assay system at the region of the test line. The system includes a portion of the gE protein (gE) (blue star) that is adsorbed to a test strip surface (e.g. nitrocellulose membrane) and another gE protein that is linked to a label (e.g., latex) (red circle) creating a gE-latex complex.

FIG. 5B illustrates a cartoon of an example sandwich assay system at the region of the control line. For example, as depicted in FIG. 513, the system includes an anti-chicken IgY (black triangle) that is adsorbed to a surface and chicken IgY that is linked to a label (e.g., latex) (red circle). The black triangle is striped as the control line and the Conjugate Pad is striped with the Chicken IgY-Latex complex, in addition to the gE-latex complex. Once the sample solution is wicked through the region of the control line the Chicken IgY-Latex complex will bind to the anti-chicken IgY on the surface. The test line region will turn a visible color (e.g. red) after enough of the complexes are bound.

FIG. 6A illustrates a graph depicting Axxin test line (TL) peak versus ELISA index value.

FIG. 6B illustrates a graph depicting Axxin intensity versus visual grades.

FIG. 7A depicts a table comparing antibody detection using an ELISA assay versus an example lateral flow assay (LFA) in accordance with the present disclosure.

FIG. 7B depicts a table displaying calculated sensitivity, specificity, false negative, and false positive rates based on the results determined in FIG. 7A.

FIG. 8A illustrates an example embodiment of an LFA when used with a blank sample depicting a sample area, a conjugate region, a test region, and a control region in accordance with example aspects of embodiments of the present disclosure.

FIG. 8B illustrates an example embodiment of an LFA when used with a positive sample depicting a sample area, a conjugate region, a test region, and a control region in accordance with example aspects of embodiments of the present disclosure.

FIG. 8C illustrates an example embodiment of an LFA used with a positive sample.

FIG. 9 illustrates a flow chart depicting the steps involved in detecting the presence or absence of an immune response in a biological sample using an example embodiment of an LFA in accordance with the present disclosure.

FIG. 10 is a slide resulting from gel electrophoresis (SDS-PAGE, or sodium dodecyl sulfate-polyacrylamide gel electrophoresis) of VZV TgE expression in CHO cells. Expected results are ~46 kDa for TgE. Lane 1: Protein Marker, (Bio-Rad Cat. No. 1610374); Lane 2: 5.00 μg truncated VZV gE under reducing conditions; Lane 3: 5.00 μg truncated VZV gE under non-reducing conditions.

FIG. 11 is an SEC-HPLC analysis of the truncated VZV gE, showing a purity of 96.58%.

### DETAILED DESCRIPTION

In one embodiment, a device useful in lateral flow assays for the detection of antibodies against Varicella-Zoster Virus (VZV) gE human serum/plasma or whole blood is disclosed. The blood sample can be obtained, for example, using venipuncture or finger stick. The assay can be used, for example, by trained professionals in a clinical laboratory, hospital setting or a physician's office, or by individual patients at home, using a finger stick to obtain the blood sample.

The isolated peptides used in the assays (i.e., SEQ ID Nos. 1 and 2, and an analogs thereof) can be produced, for example, by expressing individual glycoprotein genes. The expression can be performed, for example, in baculovirus or CHO cells, and the peptides so produced can be isolated and purified.

In some embodiments, the LFA assay can also include a control line, and the primary function of the control line is to verify that the assay worked, i.e., that fluid, such as blood, plasma, or sera, flowed through the test strip and reached the control line. It is possible to use any colored particle bound to a compound that binds to any substance on the control line, such that the binding of the compound to the substance provides a colored signal on the control line, except that one cannot use particles bound to any compound that would also bind to the substance in the test line. In one embodiment, a colored particle is bound to anti-chicken IgY, and this anti-chicken IgY binds to chicken IgY present in the control line. Alternatively, a colored particle can be bound to chicken IgY, and this chicken IgY binds to anti-chicken IgY present in the control line. However, any control can be used where a first component (a label attached to a binding partner) is present in the assay before the control line, such as in the conjugation region, and migrates along the test strip with the other components in the assay, and the component binds to a second component, which second component is bound to the control line.

In some embodiments, the individual glycoproteins are available commercially, as are monoclonal antibodies and seropositive samples for each. The use of seropositive samples can serve as a reference control. That is, if a lab has a series of patient samples, and seropositive samples are mixed into the patient samples, if the lab fails to identify a known seropositive sample as a positive test, this will ideally alert the lab that the test is not being performed correctly.

In some embodiments, the assay is based on visual labels, conjugated to the isolated peptides, and interpreted without the use of a reader system. In other embodiments, the assay is quantitative, and is designed for use with a reader system. Multiple colored labels are available for evaluation, including colloidal gold, colored latex and cellulose nanobeads. In one non-limiting embodiment, the particles have a diameter of approximately 0.4 nm, and in one aspect of this embodiment, the particles are red latex particles. Other particle sizes can certainly be used, though the particles typically have a size less than 100 nm, and a size of at least approximately 0.2 nm. These particles, and their sizes, can be selected based on the requirements of the system for sensitivity and specificity.

Representative biological sample types include whole blood, obtained by venipuncture or finger stick, plasma, or serum.

In some embodiments, the assays are in a “sandwich” format using a colored signal reagent (in one aspect of these embodiments, red latex particles). The assay can detect, at a minimum, a patient's IgG response, but in some embodiments, can also detect a patients IgM response. This is important, as it may take a substantial amount of time for a patient to produce IgG, and the ability to detect IgM means that a patient may be able to be treated that much sooner, rather than waiting until IgG is present.

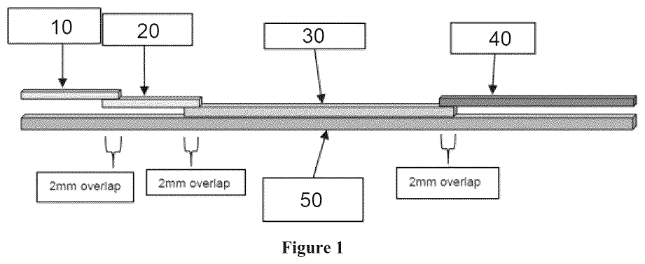

In one embodiment, as shown in FIG. 1, the assay strip includes a sample pad (10), a conjugate pad (20), a membrane (30) (e.g. a nitrocellulose membrane), and a wick pad (40), each of which is applied to a backing card (50). As shown in FIG. 1, one representative overlap between the various components is around 2 mm of overlap, though greater and lesser amounts of overlap can be used, so long as they do not interfere with the assay performance.

In some embodiments, the assay strips are contained in a standard high-performance plastic cassette, such as those available to DCN, and in other embodiments, a custom cassette is used, for example, to optimize performance.

In some embodiments, a chase or running buffer is used to facilitate the movement of the patient sample through the assay strip.

The overall system works by applying the patient sample to the sample pad, for example, in a well (not shown in FIG. 1, but typically present in a cassette into which the test strip is placed), whereby capillary forces and the wick pad aid the movement of the sample through the membrane. The sample fluid travels into the conjugate pad where labeled conjugate proteins interact with the antibodies in the patient sample, if present. In one embodiment, the labeled conjugate proteins comprise the isolated peptides of SEQ ID Nos. 1 or 2, or analogs thereof, bound, chemically or physically, to a particle, such as a colored latex or colloidal gold particle.

Then, the fluid travels over the test line on the membrane. The test line includes the VZV gE protein (i.e., the isolated peptides of SEQ ID Nos. 1 or 2, or analogs thereof) that has already been striped and dried on the membrane, and, optionally, attached to the membrane, for example, chemically or physically attached to the membrane.

If a patient sample includes a VZV antibody, the antibody will bind to the labeled conjugate protein (i.e., the isolated peptide bound to the colored particle), and the antibody will further bind to the isolated peptide present on, and, optionally, bound to, the membrane at a location corresponding to the test line. A plurality of colored particles imparts a visible signal, such as a colored line, which indicates a positive test.

That is, if positive, the patient antibodies that have bound to the isolated peptide/particle conjugates will bind to the VZV gE (in this case, the isolated peptide bound to the test strip at the test line) in a sandwich format, and the result is a visible red/brown line to be seen by the eye.

If the test is negative, no line will be visible, as no colored particles will bind (through binding of the colored particles to the isolated peptide, and the binding of the isolated peptide to the VZV antibodies) to the test line.

The sample fluid will continue to run up the membrane, past the test line and over the control line. The control line includes a substance, which in one embodiment, is an antibody or protein (e.g. anti-chicken IgY), which is already striped and dried on the membrane. The isolated peptide/particle conjugates, not bound to a VZV antibody, will have already been picked up by the sample fluid from the conjugate pad, and will attach to proteins or antibodies, such as anti-chicken IgY, on the control line. Regardless of whether the test is positive or negative, this line will show a visible line, such as a red/brown line.

The sample will run for a period of time, and, typically after the control line turns color, the result can be analyzed, in some embodiments, by a trained professional, and in other embodiments, by the patients themselves.

Embodiments described herein can be understood more readily by reference to the following detailed description and examples. Systems, compositions and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### I. Lateral Flow Assays

In general, the present disclosure is directed to lateral flow assays for detection of antibodies for VZV. Lateral flow tests (LFTs), also known as lateral flow immunochromatographic assays or rapid tests, are devices intended to detect the presence of a target substance, in this case, antibodies to VZV, in a biological sample. These tests can be used in medical diagnostics for home testing, point of care testing, or laboratory use. These tests generally show results in around five to thirty minutes.

LFTs operate on the same principles of affinity chromatography as the enzyme-linked immunosorbent assays (ELISA). In essence, these tests run the liquid sample along the surface of a pad with reactive molecules that show a visual positive or negative result. The pads are based on a series of capillary beds, such as pieces of porous paper, microstructured polymer, or sintered polymer. Each of these pads has the capacity to transport fluid (e.g., urine, blood, plasma, serum, saliva and the like) spontaneously, though, in the assays described herein, blood, plasma and serum are the primary biological fluids that are evaluated.

The sample pad acts as a sponge and holds an excess of sample fluid. Once soaked, the fluid flows to the second conjugate pad in which the manufacturer has stored freeze dried bio-active particles called conjugates (see below) in a salt-sugar matrix. The conjugate pad contains all the reagents required for an optimized chemical reaction between the target molecule (e.g., an antigen) and its chemical partner (e.g., antibody) that has been immobilized on the particle's surface. This marks target particles as they pass through the pad and continue across to the test and control lines. The test line shows a signal, often a color as in pregnancy tests. The control line contains affinity ligands which show whether the sample has flowed through and the bio-molecules in the conjugate pad are active. After passing these reaction zones, the fluid enters the final porous material, the wick, that simply acts as a waste container.

LFTs can operate as either competitive or sandwich assays.

### Colored Particles

In principle, any colored particle can be used, however latex (blue color) or nanometer-sized particles of gold (red color) are most commonly used. The gold particles are red in color due to localized surface plasmon resonance. Fluorescent or magnetic labelled particles can also be used, however these require the use of an electronic reader to assess the test result.

### Sandwich Assays

Sandwich assays are generally used for larger analytes, because they tend to have multiple binding sites. As the sample migrates through the assay, it first encounters a conjugate, which is an antibody specific to the target analyte labelled with a visual tag, such as colloidal gold or colored latex particles. The antibodies bind to the target analyte within the sample and migrate together until they reach the test line.

The test line also contains immobilized antibodies specific to the target analyte, which bind to the migrated analyte bound conjugate molecules. The test line then presents a visual change due to the concentrated visual tag, hence confirming the presence of the target molecules. In some embodiments, the sandwich assays also have a control line, which will appear whether or not the target analyte is present to ensure proper function of the lateral flow pad.

### Competitive Assays

Competitive assays are generally used for smaller analytes since smaller analytes have fewer binding sites. The sample first encounters antibodies to the target analyte labelled with a visual tag (colored particles). The test line contains the target analyte fixed to the surface. When the target analyte is absent from the sample, unbound antibody will bind to these fixed analyte molecules, meaning that a visual marker will show. Conversely, when the target analyte is present in the sample, it binds to the antibodies to prevent them binding to the fixed analyte in the test line, and thus no visual marker shows. This differs from sandwich assays in that no band means the analyte is present.

### Quantitative Tests

Most LFTs are intended to operate on a purely qualitative basis. However, in some it is possible to measure the intensity of the test line to determine the quantity of analyte in the sample. Handheld diagnostic devices known as lateral flow readers are used by several companies to provide a fully quantitative assay result. By utilizing unique wavelengths of light for illumination in conjunction with either CMOS or CCD detection technology, a signal rich image can be produced of the actual test lines. Using image processing algorithms specifically designed for a particular test type and medium, line intensities can then be correlated with analyte concentrations. One such handheld lateral flow device platform is made by Detekt Biomedical L.L.C. Alternative non-optical techniques are also able to report quantitative assays results. One such example is a magnetic immunoassay (MIA) in the LFT form also allows for getting a quantified result. Reducing variations in the capillary pumping of the sample fluid is another approach to move from qualitative to quantitative results.

### Control Line

In some embodiments, the assays will incorporate a second line which contains a further antibody (one which is not specific to the analyte) that binds some of the remaining colored particles which did not bind to the test line. This confirms that fluid has passed successfully from the sample-application pad, past the test line. By giving confirmation that the sample has had a chance to interact with the test line, this increases confidence that a visibly-unchanged test line can be interpreted as a negative result (or that a changed test line can be interpreted as a negative result in a competitive assay). That is, if the control line of the test is blank, the test is invalid.

### Blood Plasma Extraction

Because the intense red color of hemoglobin interferes with the readout of colorimetric or optical detection-based diagnostic tests, blood plasma separation is a common first step to increase diagnostic test accuracy. Plasma can be extracted from whole blood via integrated filters or via agglutination. Accordingly, in some embodiments, the test strips include an integrated blood filter.

### Speed and Simplicity

In some embodiments, the tests can take as little as a few minutes to develop. Generally, there is a trade off between time and sensitivity: more sensitive tests may take longer to develop. The other key advantage of this format of test compared to other immunoassays is the simplicity of the test, by typically requiring little or no sample or reagent preparation.

### Wells

In some embodiments, the sample pad comprises a well. In some embodiments, the well has a sufficient volume to contain a solution containing the biological sample, which sample can optionally be diluted before being placed in the well.

In certain embodiments, the volume of the well ranges from about 1 μL to about 10 μL. In certain embodiments, the volume of the well ranges from about 1 μL to about 100 μL. In certain embodiments, the volume of the well ranges from about 1 μL to about 1000 μL. In certain embodiments, the volume of the well ranges from about 1 μL to about 5000 μL. In certain embodiments, the volume of the well ranges from about 1 mL to about 10 mL. In certain embodiments, the volume of the well ranges from about 1 mL to about 100 mL. In certain embodiments, the volume of the well ranges from about 1 mL to about 1000 mL.

In some embodiments, the well is located at a position of the LFA selected from a corner, an end, a center, a junction, an off-center, and a bend of the LFA. In some embodiments, the well comprises one or more pads selected from a salt pad, a probe pad, a polymer pad, and combinations thereof. In some embodiments, the well comprises a plurality of pads. In some embodiments, the first/second phase solutions separate and/or the target analyte concentrates as it flows through the plurality of pads. In some embodiments, the first/second phase solutions separate and/or the target analyte concentrates as it flows vertically through the plurality of pads. In some embodiments, the first/second phase solutions separate and/or the target analyte concentrates as it flows vertically through the plurality of pads due to gravity. In some embodiments, the first/second phase solutions separate and/or the target analyte concentrates as it flows vertically through the plurality of pads due to capillary action. In some embodiments, the well is a paper well. In some embodiments, the paper well is a three-dimensional paper structure holds a larger volume of sample compared to a typical paper strip used in LFA. In some embodiments, the paper well is composed of paper material that allows phase separation to occur and subsequent analyte concentration in the leading fluid. In some embodiments, the flow of the leading fluid is directed toward the absorbance pad that enables analyte detection.

In some embodiments, the device utilizes a “concentration-as-it-flows” mechanism, while further accelerating the flow and macroscopic phase separation utilizing gravitational force in the well. In some embodiments, the well provides a cross-sectional area sufficient to promote phase separation, since the first phase solution and the second phase solution may flow at a different speed due to differences in viscosity of the phase solutions, as well as differences in affinity for the paper material. In some embodiments, the well enhances or accelerates the phase separation and/or concentration of target analytes as the phase solution(s) travels through the well and emerges in the leading fluid. In some embodiments, the LFA test strip is connected directly to the well in a downstream position, so the concentrated analytes in the leading fluid first come in contact with the LFA strip and the detection step occurs concurrently with the concentration process, further reducing the overall assay time.

### LFA Design/Architecture

In some embodiments, the LFA strip has a width that does not vary from a first end to a second end. In some embodiments, the width is defined as a dimension perpendicular to the direction of flow within the LFA and in a plane of the length. In some embodiments, a first portion of the LFA strip has a first width and the second portion of the LFA strip has a second width, where the first width and the second width are different. In some embodiments, the first width is greater than the second width, while in other embodiments, the first width is less than the second width. In certain embodiments, it is contemplated that the LFA strip comprises more than two widths, e.g., the strip may continuously narrow, or may show progressive narrowing at three or more locations. In some embodiments, the first portion comprises the sample pad and the second portion comprises the detection zone. In some embodiments, a wider sample pad segment allows more target analyte in the sample to bind to the probe compared to an LFA strip wherein the width of the LFA strip does not vary. In some embodiments, a wider sample pad segment allows a greater volume of sample, and thus, more target analyte, to bind to the probe compared to an LFA strip wherein the width of the LFA strip does not vary.

In some embodiments, the LFA comprises a slope (e.g. a change in depth of the LFA along the length of the LFA). In some embodiments, wherein the LFA does not comprise a slope, a portion of the probe-analyte complex is left in the sample pad. In some embodiments, wherein the LFA comprises a slope, more probe analyte complex flows through the LFA than an LFA without a slope.

In some embodiments, the LFA is designed to be used with a probe that comprises or complexes with a magnetic/paramagnetic particle. In some embodiments, the LFA comprises a paper strip with a fork at the end of paper strip used to split the flow of the ATPS first and second phase solution. In some embodiments, the LFA detection zone is located on a prong of the fork, and magnets are located near or at the prong. The magnets concentrate the probe/probe-analyte complex into the fluid flowing into the LFA detection zone, which results in increased sensitivity of the diagnostic. Conversely, in some embodiments, the probe comprises the magnet or magnetic field, and the device comprises a magnetic particle or paramagnetic particle that is located near or at the prong.

In some embodiments, the LFA comprises a 3D architecture. In some embodiments, the LFA comprises layers of porous matrix resulting in a 3D architecture. In some embodiments, the 3D architecture integrates the ATPS with the LFA. In some embodiments, the ATPS has a long phase separation time, (e.g. a micellar ATPS) and phase separation time is improved by using a 3D architecture (e.g. increasing the height of the LFA strip). In some embodiments, the mixed phase solution separates into the first phase solution and the second phase solution as the phase solutions flow vertically through the LFA (e.g. through the layers of porous matrix).

In some embodiments, the LFA has a thickness (e.g. height, depth or vertical dimension). In some embodiments, the thickness is about 0.1 tutu to about 30 cm. In some embodiments, the thickness is about 0.1 mm to about 1 mm, about 0.1 mm to about 10 mm, or about 0.1 mm to about 1 cm. In some embodiments, the thickness is about 1 mm to about 10 mm, about 1 mm to about 1 cm, about 1 mm to about 1.5 cm, about 1 mm to about 3 cm, about 1 mm to about 3.5 cm, about 1 mm to about 4 cm, about 1 mm to about 4.5 cm, about 1 mm to about 5 cm, about 1 mm to about 5.5 cm, about 1 mm to about 6 cm, about 1 mm to about 6.5 cm, about 1 mm to about 7 cm about 1 mm to about 7.5 cm, about 1 mm to about 8 cm, about 1 mm to about 8.5 cm, about 1 mm to about 9 cm, or about 1 umm to about 9.5 cm, or about 1 mm to about 10 cm. In some embodiments, the thickness is about 0.5 cm to about 5 cm.

### Isolated Peptides

In some embodiments, the assays described herein include a solid support having at least two regions, each region defining an area of the solid support that includes an isolated peptide. The isolated peptides are described in more detail below.

In some embodiments, the isolated peptide can include an amino acid sequence which comprises a portion of the gE protein. Representative, non-limiting amino acid sequences include:

(SEQ ID 1)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHVHHHHHH,
(SEQ ID 2)
MGWSCIILLFLVATATGVHSSVLRYDDFHIDEDKLDTNSVYEPYYHSDHA
ESSWVNRGESSRKAYDHNSPYIWPRNDYDGFLENAHEHHGVYNQGRGIDS
GERLMQPTQMSAQEDLGDDTGIHVIPTLNGDDRHKIVNVDQRQYGDVFKG
DLNPKPQGQRLIEVSVEENHPFTLRAPIQRIYGVRYTETWSFLPSLTCTG
DAAPAIQHICLKHTTCFQDVVVDVDCAENTKEDQLAEISYRFQGKKEADQ
PWIVVNTSTLFDELELDPPEIEPGVLKVLRTEKQYLGVYIWNMRGSDGTS
TYATFLVTWKGDEKTRNPTPAVTPQPRGAEFHMWNYHSHV,

and analogs thereof

In some embodiments, the first isolated peptide and the second isolated peptide used in the assays can consist substantially of the amino acid sequence set forth as SEQ ID 1 or 2. As used herein, “consisting substantially of” an amino acid sequence indicates that the isolated peptide includes the stated amino acid sequence with 5 or fewer amino acid additions or deletions to the N-terminus and/or the C-terminus. Thus, for SEQ ID 1, which includes 326 amino acids, the isolated peptide can have an amino acid sequence that includes no less than 316 amino acids and no greater than 336 amino acids.

The isolated peptide can include an amino acid sequence (e.g., SEQ ID 1) that may further include one or more linker regions for binding the peptide to a region of the solid support. For instance, the one or more linker regions may include oligomers such as polyethylene glycol, acrylates, and/or peptides. Additionally or alternatively, the linker region may include a chemical modification such as thiolation, amidation, and/or esterification, which may be used to chemically attach the isolated peptide to the solid support.

The isolated peptide sequences provided herein (e.g., SEQ ID NOs: 1-2) may also have 1, 2, or 3 conservative mutations. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In another embodiment, the isolated peptide sequences are analogs of SEQ ID Nos. 1-2, which have at least 90% or greater sequence homology to any one or more of the polypeptide sequences of SEQ ID Nos. 1-2. More preferably, the peptide sequences have at least 95% or greater sequence homology, even more preferably at least 98% or greater sequence homology, and still more preferably at least 99% or greater sequence homology to any one or more of SEQ ID Nos. 1-2.

Methods for determining homology between nucleic acid and amino acid sequences are well known to those of ordinary skill in the art. For example, the “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Conjugates of the Isolated Peptide to Particles and/or Solid Supports

In one embodiment, the isolated peptide is bound to a particle, such as a latex or a gold particle, and in some aspects of this embodiment, the isolated peptide is chemically or physically bound to the particle, for example, covalently bonded, ionically bonded, bonded by chemisorption or physisorption, bonded via functional linking groups or linker molecules, or bonded via hydrogen bonding, hydrophobic interaction, adsorption, absorption, or Van Der Waals attraction.

In another embodiment, the isolated peptide is bound to a substrate, and in some aspects of this embodiment, the isolated peptide is chemically or physically bound to the substrate, for example, covalently bonded, ionically bonded, bonded by chemisorption or physisorption, bonded via functional linking groups or linker molecules, or bonded via hydrogen bonding, hydrophobic interaction, adsorption, absorption, or Van Der Waals attraction. In one embodiment, where the isolated peptide is bound to a substrate, the location where the isolated peptide is bound to the substrate is at the test line of an LFA.

In another embodiment, the isolated peptide is bound to a particle, and also bound to a VZV antibody. In one aspect of this embodiment, the antibody in the “isolated peptide-particle-antibody conjugate” is also bound to an isolated peptide bound to a solid support. The conjugate (or complex) of the VZV antibody to a) the isolated peptide bound to a particle, and b) the isolated peptide bound to a solid support, is referred to as a “sandwich,” and is used in the sandwich assays described herein to indicate a user has tested positive for VZV infection.

### Preparing Conjugates of GNPs and the Isolated Peptides

In some embodiments, rather than using latex particles, gold nanoparticles (GNPs) are used. Gold nanoparticles can be prepared to result in a clear, cherry-colored solution with particle sizes under 100 nm, such as around 25-30 nm in diameter. To prepare the GNPs, a solution of anti-Ge antibody can be incubated with a colloidal gold solution for a period of time, such as around 30 min, followed by the addition of thiolated-PEG5000, using a molar ratio of 3000:1 for PEG:GNP and an additional incubation of 30 min. To prevent nonspecific binding of other proteins to the surfaces of the colloidal gold, a bovine serum albumin (BSA) solution can be added to the mixture and mixed for an additional period of time, such as around 10 mir. The resulting solution can be gently mixed during the incubation period. To remove free (unbound) antibodies, PEG, and BSA, the mixture can be subsequently centrifuged for an additional period of time, typically around 30 min, at a temperature of around 4 °C and 9,000 g.

The resulting pellet of GNPs can be washed, for example, with a 1% BSA solution. Finally, the recovered GNPs can be resuspended in a suitable buffer, such as a 0.1 M sodium borate buffer at a pH of around 9.

### Solid Supports

Solid supports used in LFAs are well known to those of skill in the art. In some embodiments, the solid supports are housed, for example, in a cassette, such as a plastic frame surrounding the test strip, with a well for the biological sample to be placed, and a window through which the two lines can be seen.

In some embodiments, the solid supports can include one of the at least two regions having the isolated peptide linked to the solid support. For instance, the region having the isolated peptide linked to the solid support can include the isolated peptide covalently or non-covalently bound to the solid support.

In some embodiments, the solid support includes one or more chemical functionalities which can be used to covalently link the solid support to one or more isolated peptides. For example, where the isolated peptides include a linker, the chemical functionalities on the solid support can be reacted with the linker region to bind the isolated peptide to the solid support.

Another aspect of some example solid supports can include one of the at least two regions having the isolated peptide not linked to the solid support. More particularly, the region having the isolated peptide not linked to the solid support can include the isolated peptide in contact with the solid support such that the isolated peptide can be solubilized when contacted with a liquid medium. For instance, the isolated peptide may be dried onto the solid support so that when the liquid medium is applied, some or all of the isolated peptide is solubilized in the liquid medium. Representative liquid media include various aqueous media such as water and salt/saline solutions. Additionally, some liquid media may include bodily fluids such as whole blood, blood serum, and/or lymph drainage.

For certain implementations, the isolated peptide not linked to the solid support can be fused to a heterologous protein, a detectable agent, a reactive agent, or combinations thereof. For example, the isolated peptide not linked to the solid support can include one or more functionalities to produce a detectable signal when incorporated as an immune assay. More particularly, in some implementations, the isolated peptide not linked to the solid support can include a heterologous protein that can act as or bind to a control marker. In this manner, as the isolated peptide not linked to the solid support is solubilized, it can bind to the control marker to indicate that the assay is working properly.

To produce a signal, the isolated peptide can also include a detectable agent and/or a reactive agent. The detectable agent can include an inert material such as a colored latex bead, colloidal metal (e.g., gold, silver, etc.), or other suitable products that upon binding to a region of the solid support, become concentrated such that visible signal is produced. The reactive agent can include a non-inert material that is chemically modified to produce a detectable signal such as fluorescence or a color change.

As an example for illustration, the isolated peptide not linked to the solid support can include a portion of the gE protein sequence (e.g., SEQ ID 1) that is fused to Biotin, Avidin, and/or Streptavidin. The Biotin-Avidin system can further be linked to a detectible agent such as a colored latex bead, colloidal gold particle, and the like. In this manner, the isolated peptide not linked to the solid support includes a detectible agent linked to the amino acid sequence via a heterologous protein (e.g., Avidin or Streptavidin).

In some example implementations, the isolated peptide not linked to the solid support can have a concentration of no less than 10 ng/mm² and no greater than 600 ng/mm² based on the area of the region.

Additionally or alternatively, in certain example implementations, the isolated peptide linked to the solid support can have a concentration of no less than 1 ng/mm² and no greater than 500 ng/mm² based on the area of the region. For instance, the isolated peptide linked to the solid support can have a concentration of no less than 50 ng/mm² and no greater than 450 ng/mm², no less than 80 ng/mm² and no greater than 400 ng/mm², no less than 100 ng/mm² and no greater than 350 ng/mm², or no less than 120 ng/mm² and no greater than 350 ng/mm², based on the area of the region.

Solid Supports Comprising Isolated Peptides Rather than Bound Antibodies

In some aspects, the antibody detection assays include a solid support that is not directly linked to an antibody. While detecting antibodies is a primary use of certain embodiments, in other embodiments, it is not necessary to incorporate antibodies in the assays (e.g., via attachment to the solid support). In some aspects, the solid supports can include two regions which have an isolated peptide in accordance with the amino acid sequences disclosed herein. Further, the isolated peptide in each region may include the same amino acid sequence (e.g., both include one of SEQ ID 1 or 2) or the isolated peptide in each region may include different amino acid sequences (e.g., one includes SEQ ID 1 and the second includes SEQ ID 2, and all other combinations of these sequences).

### Solid Supports Comprising a Control Region

In other embodiments, the antibody detection assays include a solid support that further includes a control region. The control region can include a control agent that acts to bind to a portion of the isolated peptide not linked to the solid support.

In some aspects of these embodiments, the control region can include a protein that can dimerize or otherwise interact with the heterologous protein present on the isolated peptide not linked to the support.

In one representative example, the control agent can include Avidin, which may dimerize and/or bind to the Biotin-Avidin system to produce accumulation of the detectible agent in the control region.

As another example, the control agent can include a protein, such as an antibody (e.g., IgG, IgY, IgM, etc.) or a target of an antibody (e.g., anti-IgG, anti-IgY, anti-IgM, etc.) For instance, the isolated peptide not linked to the solid support can include the antibody (e.g., chicken IgY) bound directly to the isolated peptide as a fusion protein, or may include the antibody bound to the detectible agent, and the control region can include the target corresponding to the antibody (e.g., anti-chicken IgY).

Alternatively, the isolated peptide not linked to the solid support can include the target (e.g., anti-chicken IgY) bound directly to the isolated peptide as a fusion protein, or may include the target bound to the detectible agent, and the control region can include the antibody corresponding to the target (e.g., chicken IgY).

As should be understood various combinations of the preceding disclosure may be produced and are within the scope of the present application. For instance, one example implementation includes a solid support that includes: a wicking material for directing material flow along a direction of the solid support; a sample region comprising a first isolated peptide, wherein the first isolated peptide is not linked to the solid support, and wherein the first isolated peptide is fused to a heterologous protein; a detectable agent, a reactive agent, or combinations thereof; a test region positioned beyond the sample region along the direction of material flow, wherein the test region comprises a second isolated peptide linked to the solid support; and a control region positioned beyond the test region along the direction of material flow, wherein the control region comprises a control agent, and wherein each of the first isolated peptide and the second isolated peptide comprises an amino acid sequence set forth as SEQ ID 1.

### II. Methods of Detecting VZV Antibodies

In another embodiment, methods for detecting anti-Varicella-Zoster Virus (VZV) antibodies in a biological sample containing antibodies are disclosed. The methods involve: contacting the biological sample with a solid support including an isolated peptide, wherein the isolated peptide includes an amino acid sequence set forth as: (SEQ ID 1), and where contacting is performed under conditions sufficient to form an immune complex between the isolated peptide and an antibody present in the biological sample. The presence or absence of the immune complex can then be detected, preferably visually, though other suitable methods may be used. Detection of the immune complex provides indication of the presence of anti-VZV antibodies in the biological sample.

In one aspect, the methods involve detecting the presence or absence of an immune complex based on the presence of a color change on the solid support (e.g., the formation of a red, yellow or other colored shape such as a line).

In another aspect, the methods involve incubating the assay after contacting the biological sample with the solid support for a given time period. The incubation time is generally less than about 1 hour, such as no less than 0.5 minutes and no greater than 60 minutes, no less than 1 minutes and no greater than 55 minutes, no less than 5 minutes and no greater than 50 minutes, no less than 10 minutes and no greater than 50 minutes, no less than 15 minutes and no greater than 45 minutes, or no less than 20 minutes and no greater than 40 minutes.

### III. Kits

In some embodiments, the assay may be in the form of a kit. The kit may include other reagents or materials for facilitating diagnosis such as a chase buffer to facilitate movement of the sample along the strip, a result indicator to facilitate interpretation of the assay, and/or a needle or stick to facilitate sample collection. The kit may also include pertinent information for obtaining a blood sample, performing the assay using the blood sample, and interpreting the results of the assay. In addition to these elements, the kit can include a capillary tube to draw blood from the sample in a measured amount for adding to the well of the LFA. The kit can also include a chase buffer to add to the blood sample in the well. A chase buffer is typically a salt-based buffer, such as physiological saline and phosphate buffered saline.

### IV. Theranostic Applications

In some embodiments, once a patient has been diagnosed as having a Varicella Zoster infection, the patient is then treated with an antiviral medication. Although other agents can be used, several agents commonly used against VZV, and which can be used in the theranostic methods described herein, are discussed below.

Whether or not a patient tests positive, the patient can also benefit from prophylaxis with a Shingles vaccine. Accordingly, in one aspect of this embodiment, the patient is encouraged to take a Shingles vaccine regardless of their testing status, and, in some aspects, are administered a Shingles vaccine.

### Acyclovir and Valacvclovir

Acyclovir, an acyclic analogue of guanosine, is a selective inhibitor of VZV and HSV replication. The drug is converted to acyclovir triphosphate in vivo, and acyclovir triphosphate inhibits viral DNA synthesis by competing with deoxyguanosine triphosphate as a substrate for viral DNA polymerase. The median inhibitory concentration of acyclovir necessary to reduce VZV plaque counts by 50% (IC₅₀) is approximately 3 μg/ml, and this is commonly achieved using oral administration of multiple doses of 200 mg or 800 mg of acyclovir, which provide mean plasma peak concentrations at steady state are approximately 0.6 and 1.6 μg/ml, respectively.

Valacyclovir is an orally administered prodrug of acyclovir (i.e., the L-valine ester of acyclovir), that overcomes the problem of poor oral bioavailability and exhibits improved pharmacokinetic properties. Using this prodrug formulation, the bioavailability of acyclovir is increased to about 54%, yielding peak plasma acyclovir concentrations that are three- to fivefold higher than those achieved with oral administration of the parent compound. Oral valacyclovir doses of 500 mg or 1000 mg produce peak plasma acyclovir concentrations of 3-4 and 5-6 μg/ml, respectively. Following administration of valacyclovir at a dose of 2 g orally four times daily, plasma acyclovir area-under-the-curve (AUC) values approximate those produced by acyclovir given intravenously at a dose of 10 mg/kg every 8 hours.

Acyclovir is available in topical, oral, and intravenous formulations. The dermatologic preparation consists of 5% acyclovir in a cream or polyethylene glycol ointment base. Topical acyclovir is intended for treatment of minor mucocutaneous HSV infections and plays no role in treatment of VZV. Oral acyclovir preparations include a 200 mg capsule, 400 and 800 mg tablets, and a liquid suspension (200 mg per 5 ml). Acyclovir sodium for intravenous infusion is supplied as a sterile water-soluble powder that must be reconstituted and diluted to a concentration of 50 mg/ml. The approved dose of oral acyclovir for chickenpox is 200 mg/kg (up to a maximum of 800 mg) 4-5 times daily for 5 days. Adults with herpes zoster can be treated with oral acyclovir at a dose of 800 mg five times daily. The recommended dose of intravenous acyclovir for VZV infections is 10 mg/kg every 8 hours, although higher doses (12-15 mg/kg) are sometimes used for life-threatening infections, especially in immunocompromised patients. Dosage reduction is required in patients with renal insufficiency. Valacyclovir is available as 500 mg and 1000 mg tablets. The recommended dose for immunocompetent adults with varicella or herpes zoster is 1000 mg three times daily for 7 days.

### Penciclovir and Famciclovir

Penciclovir is an acyclic guanine derivative that resembles acyclovir in chemical structure, mechanism of action, and spectrum of antiviral activity. Like acyclovir, penciclovir is first monophosphorylated by viral TK, then further modified to the triphosphate form by cellular enzymes. Penciclovir triphosphate blocks viral DNA synthesis through competitive inhibition of viral DNA polymerase. Unlike acyclovir triphosphate, penciclovir triphosphate is not an obligate chain terminator and can be incorporated into the extending DNA chain. Because penciclovir is very poorly absorbed, famciclovir (the diacetyl ester of 6-deoxy-penciclovir) was developed as the oral formulation. The first acetyl side chain of famciclovir is cleaved by esterases found in the intestinal wall and the second acetyl group is removed on first pass through the liver. Oxidation catalyzed by aldehyde oxidase occurs at the six position, yielding penciclovir.

When administered as the famciclovir prodrug, the bioavailability of penciclovir is about 77%. Following a single oral dose of 250 mg or 500 mg of famciclovir, peak plasma penciclovir concentrations of 1.9 and 3.5 μg/ml are achieved at 1 hour. The pharmacokinetics of penciclovir are linear and dose dependent over a famciclovir dosing range of 125-750 mg. Famciclovir is available as 125 mg, 250 mg, and 500 mg tablets. In the United States, the recommended dose of famciclovir for uncomplicated herpes zoster is 500 mg three times daily. Famciclovir doses of 250 mg three times daily and 750 mg once daily are approved for treatment of shingles in some countries.

### Brivudin

Brivudin (bromovinyl deoxyuridine) is a highly potent thymidine nucleoside analogue with selective activity against VZV, and which functions by inhibiting the viral DNA polymerase. The drug is available in several countries as a 125 mg tablet.

### Foscarnet

Foscarnet (phosphonoformic acid) is a pyrophosphate analogue that functions as an inhibitor of viral DNA polymerase by blocking the pyrophosphate binding site. Foscarnet is administered intravenously, at doses ranging from 40 mg/kg every 8 hours to 100 mg/kg every 12 hours.

### Vidarabine

Vidarabine (adenine arabinoside) is an intravenous antiviral drug, and is effective for VZV infections in immunocompromised patients.

### Interferon

Administration of alpha-interferon to immunocompromised patients with herpes zoster reduces the risk of viral dissemination, but has little impact on dermatomal rash healing or pain.

### V. Representative Antibody Detection Assays

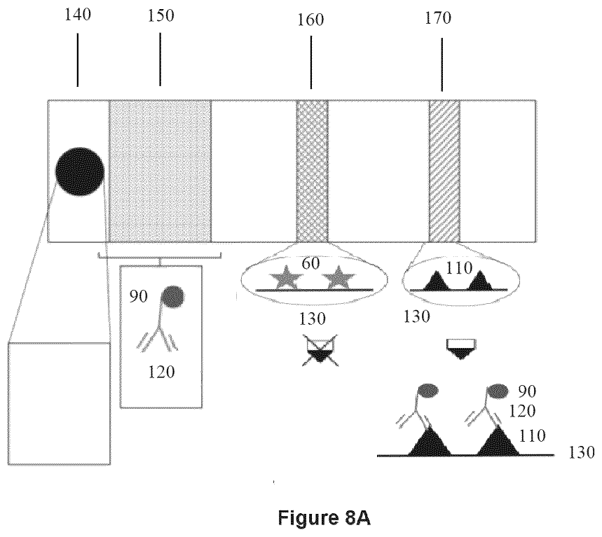

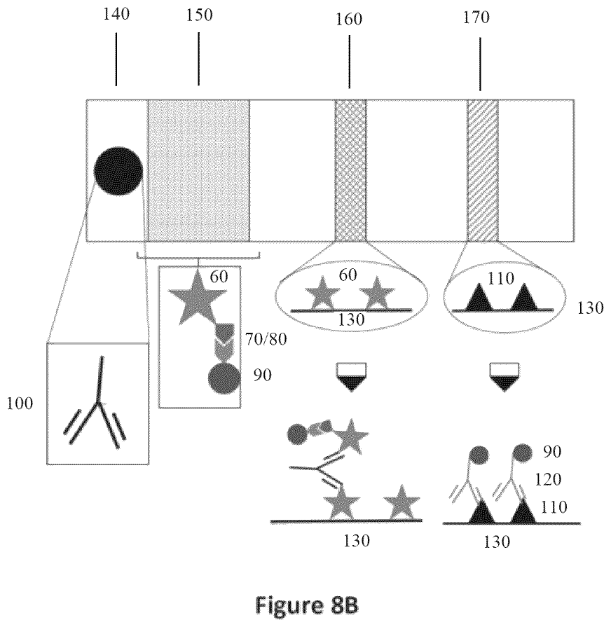

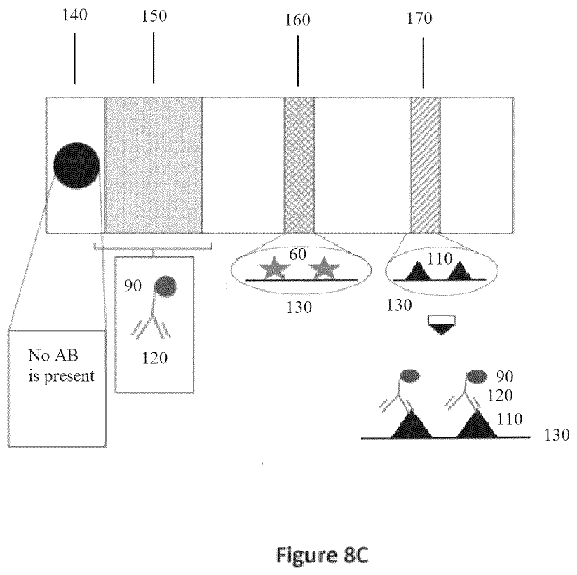

With reference now to the Drawings, the present disclosure contemplates antibody detection assays that can be formed on a solid support. The solid support can include two or more regions which include an isolated peptide. For instance, an example solid support can include a backing card on which the assay is formed. The assay can include a sample region for applying a biological sample, optionally, a membrane region for reducing unnecessary components in the biological sample (e.g., cells), a binding region which includes the isolated peptide not bound to the solid support for creating an immune complex with antibody present in the biological sample, and a test region which includes the isolated peptide bound to the solid support. As the soluble immune complex migrates to the test region, a sandwich capture causes the immune complex to concentrate at the test region to form a detectable signal. FIG. 1 illustrates one example design for a solid support. The solid support includes a blood separator pad for the sample region (1-30 mm MF1 or other suitable material (which is shown as part of well (10) in FIG. 1, and the sample area (identified as “140” in FIG. 8A), a binding region (1-30 mm 6614). The solid support also includes a membrane region (3-50 mm CN95 or other suitable material), and a test region (1-20 mm Ahlstrom 222 or other suitable material). The overall layout can be organized so that fluid applied to the sample region migrates laterally to the binding region prior to reaching the test region. Fluid migration allows soluble complexes such as the immune complex to reach the test region and provide a detectable signal. As should be understood, numbers illustrated in FIG. 1 are for example purposes only and need not constrain the size of each region.

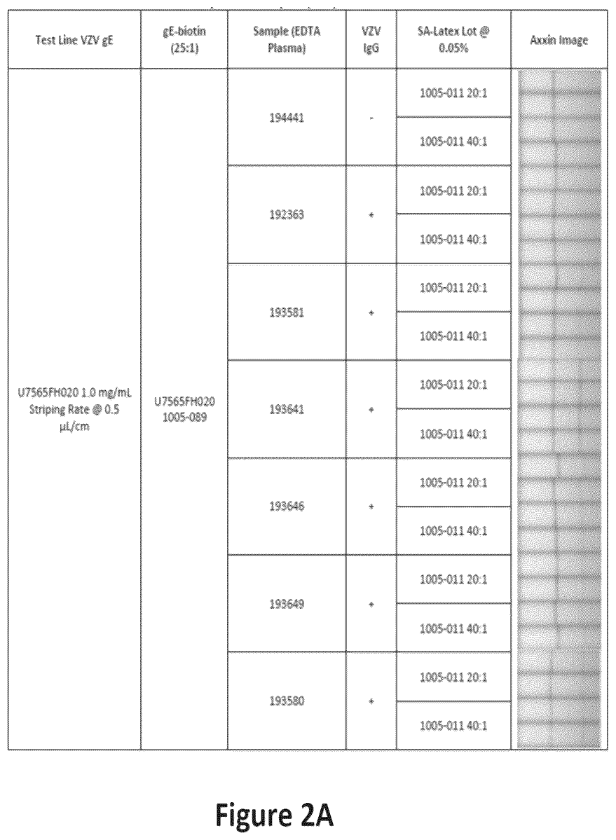

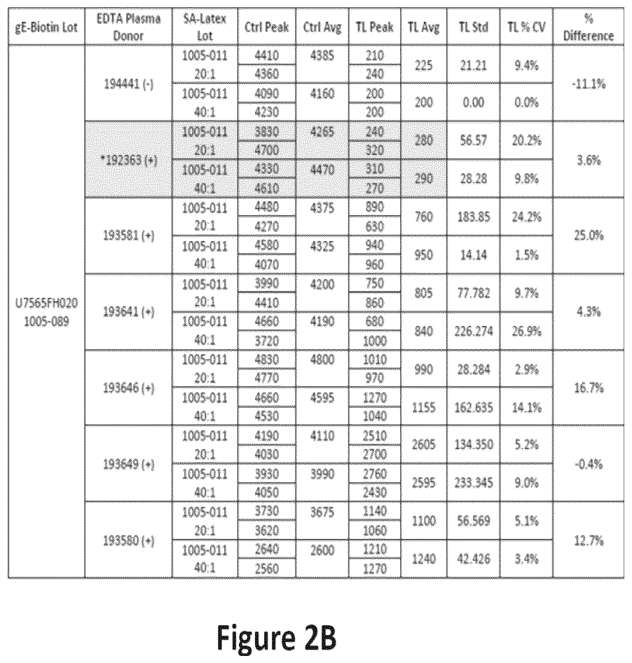

Referring now to FIG. 2A, an example test strip system was used to characterize the presence of VZV antibodies in plasma samples from different patients. At the test region (test line), the isolated peptide was applied at a stripping rate of 0.5 μL/cm at a concentration of 1.0 mg/mL. At the binding region, the ratio of the VZV amino acid sequence to biotin (gE-biotin) is 25:1. Seven different plasma samples (194441, 192363, 193581, 193641, 193646, 193649, 193—549, and 193580) were run in duplicate and images taken using Axxin imager for strips including unbound isolated peptide and latex complex at a ratio of 20:1 or 40:1 gE, latex bead to peptide. The Axxin images display clear control signal (left line) in all images and a test line (right line) depending on whether the sample plasma includes VZV IgG.

The results of FIG. 2A are quantified in FIG. 2B. As shown, the two ratios have similar TL average values (all samples were determined to be within 25% and most were within 15%).

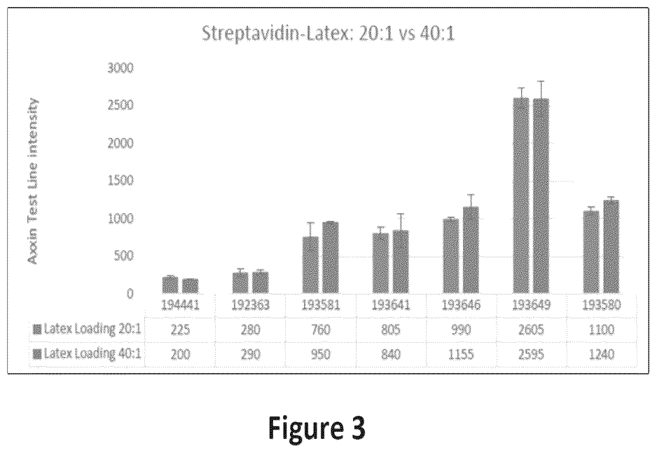

Referring now to FIG. 3, this figure depicts a bar graph summarizing the data in FIGS. 2A and 2B. As shown, the different latex loadings (20:1 or 40:1) display similar test line intensity, indicating that various latex loadings could be used to produce a VZV antibody test assay.

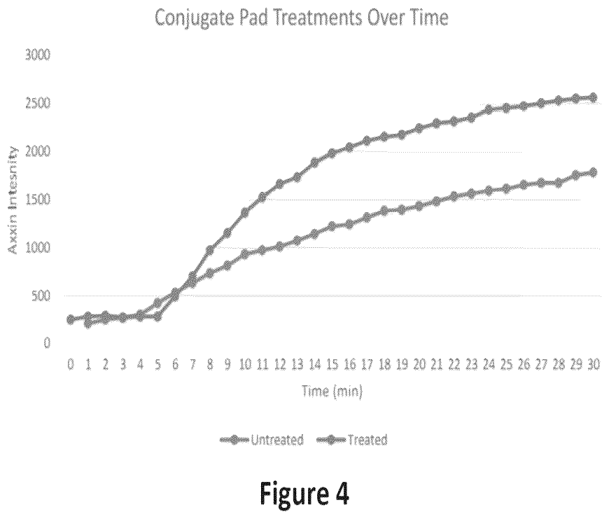

Referring now to FIG. 4, this figure depicts a graph displaying Axxin intensity measurements for an example test system taken at different time points. After 30 minutes, the signal intensity for both untreated and treated test systems are above a threshold intensity (e.g., 1000). The threshold intensity can be determined using standard curves, or may be based relative to the starting intensity. For both untreated and treated systems the initial intensity is about 250 units. Additionally, both the untreated and treated systems, reach an intensity reading of 1000, which is 300% greater than the initial intensity, within 15 minutes of applying the sample.

One aspect of some example test assays can include a treatment to reduce fluid wicking which would allow for increased interaction time between antibodies and the isolated peptide complex. As shown in FIG. 4, the increased interaction time produces an increase in signal intensity for time points greater than 6 minutes. Thus, for samples having lower antibody concentrations, it may be beneficial to use a solid substrate that includes a treated material for running the lateral flow assay to ensure. Example material treatments can include spraying or otherwise modifying the binding region of the solid support which includes the isolated peptide not bound to the solid support to also include a protein (e.g., bovine serum albumin) or other compound (e.g., Tergitol).

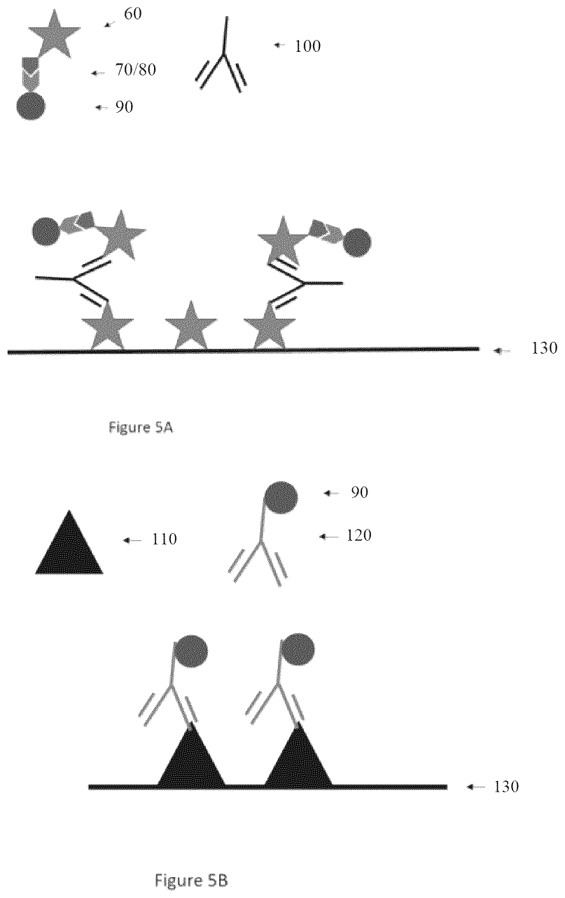

FIG. 5A illustrates a cartoon of an example sandwich assay system at the region of the test line (160). The system includes a portion of the gE protein (gE) (60, depicted as a blue star) that is adsorbed to a test strip surface (e.g. nitrocellulose membrane) and another isolated gE protein (60) that is linked to a label (e.g., a latex particle) (90, depicted as a red circle), creating a gE-latex complex. The gE and label can be linked using various polymers, peptides, chemical reactions, or other systems. For example, as depicted in FIG. 5A, the linker can include two components (e.g., Biotin and Avidin) (70 and 80, depicted as red and green connected symbols).

The Conjugate Pad (150) is striped with the gE (60)-latex (90) complex, in addition to the Chicken IgY (120)-Latex (90) complex. The assay works when the sample that contains anti-gE IgG and/or IgM (collectively referred to as (100) binds with the gE, also referred to herein as the isolated peptide of SEQ ID No. 1 or 2, or analog thereof (60)-latex (90) complex, forming an anti-gE-gE-latex complex. The sample solution is then wicked up the test strip to the region of the test line (160), where the anti-gE-gE-latex complex binds to the isolated gE peptide (60) adsorbed to the surface (130). The test line region will turn a visible color (e.g. red) after enough of the complexes are bound.

FIG. 5B illustrates a cartoon of an example sandwich assay system at the region of the control line. For example, as depicted in FIG. 5B, the system includes an anti-chicken IgY (110, depicted as a black triangle) that is adsorbed to a surface (130) and chicken IgY (120) that is linked to a label (e.g., a latex particle) (90, depicted as a red circle). The chicken IgY (120) and label (90) can be linked using various polymers, peptides, chemical reactions, or other systems. The anti-chicken IgY (110, black triangle) is striped on the control line (170) and the Conjugate Pad (150) is striped with the Chicken IgY (120)-Latex particle (90) complex, in addition to the gE (60)-latex particle (90) complex. Once the sample solution is wicked through the region of the control line (170), the Chicken IgY (120)-Latex particle (90) complex will bind to the anti-chicken IgY (110) on the surface (130). The test line region (160) and control line region (170) will turn a visible color (e.g. red) after enough of the complexes are bound.

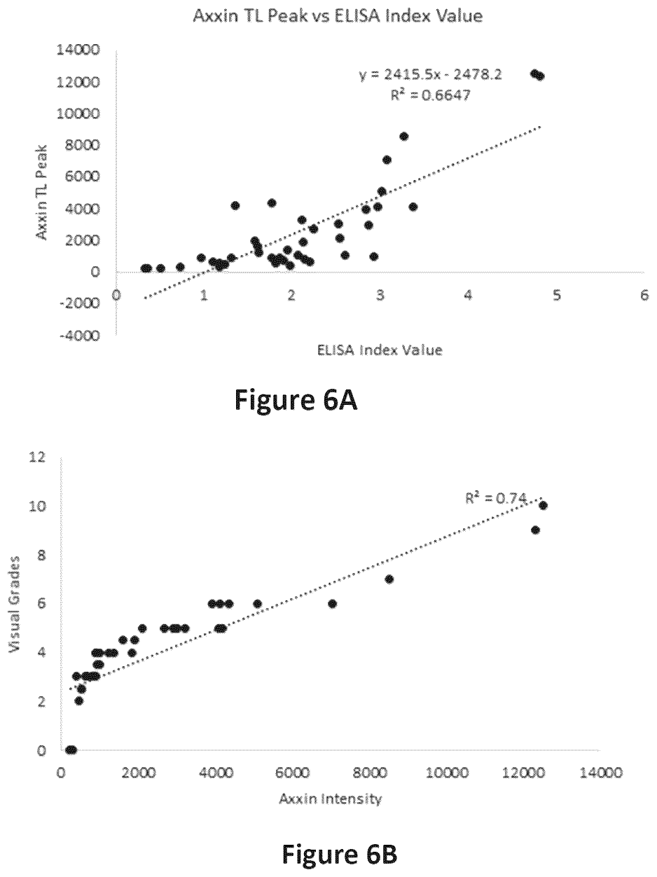

Referring now to FIG. 6A, the graph depicts a comparison between an FDA approved ELISA kit against VZV antibodies and an example LFA assay in accordance with the disclosure. As shown there is a loose correlation between the ELISA score and test line (TL) peak intensity. As shown in FIG. 6B, the loose correlation may be explained based on a non-linear correlation between Axxin signal intensity and visual grades. Even with such discrepancies, comparing the results of each testing method demonstrates that the LFA assay may be at least as sensitive as the ELISA kit.

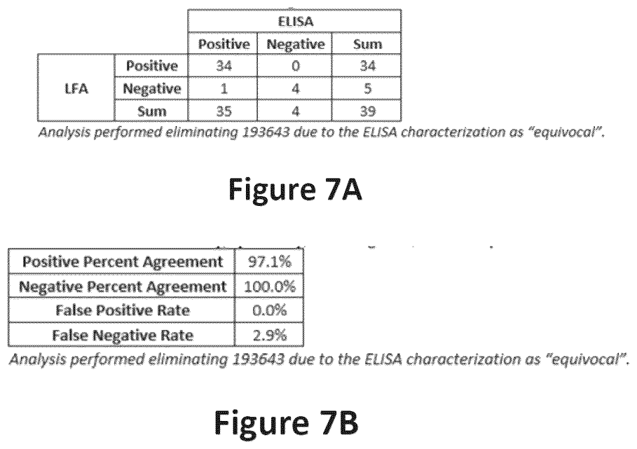

FIGS. 7A and 7B provide a quantitative assessment of the data in FIGS. 6A and 6B. As shown, classifying the assay results as positive or negative for anti-VZV antibodies yields very high agreement. Out of 39 samples, only 1 sample had different results between the ELISA assay and the LFA assay. This demonstrates that an LFA assay can be just as effective as ELISA. Further, the LFA assay may provide several advantages over other assays such as ELISA, these advantages can include simple to use with under 30 minutes read time and no need for adding additional reagents (can be self-contained and results provided at point-of-care). Additional advantages can include a testing system that can take serum, plasma, whole blood, and fingerstick blood types (ELISA is only serum). Additionally, the whole assay is stable and can be stored 2-30 °C. Further, the detection mechanism for ELISA uses viral lysate whereas the VZV LFA uses an isolated peptide including a specific amino acid sequence of the gE antigen, which can be more sensitive to binding/detection. Beyond each of these advances, ELISA is a completely different testing system that uses a second antibody (e.g., anti-human IgG) to form the sandwich complex, whereas the testing assays disclosed herein utilize a gE complex (e.g., isolated peptide that includes a detectible agent).

FIG. 8A illustrates an example embodiment of a lateral flow assay formed on a solid support (130) in accordance with the present disclosure. The figure depicts application of a blank sample (unfilled circle) onto a sample area (140) of the LFA. As the sample migrates through the conjugate region (150), which includes An isolated gE peptide (60) not linked to the solid support (130) that is fused (chemically or physically) to a particle (90), though could alternatively be fused to a heterologous protein; a detectable agent, a reactive agent, or combinations thereof. In this case the isolated gE peptide (60) is fused to a particle (90, circle), which is one example of a detectible agent, for example, via one or more heterologous proteins (cheveron(s)). The fluid migration solubilizes some of the peptide (60) not linked to the solid support (130) so that it moves up to a test region (160) which includes the peptide (60) linked to the solid support (130) depicted as a star in contact with the dark line). The sample further moves up to the control region (170), which includes a marker (110), in this case, anti-chicken IgY, bound to the solid support (130), collectively depicted as a triangle in contact with dark line). The marker (110) in the control region can be specific for binding one or more of the heterologous protein(s). In this manner, the blank sample can lead to a signal forming at the control line (170), but not forming at the test line (160).

FIG. 8B illustrates the same example embodiment of a lateral flow assay as shown in FIG. 8A. However, FIG. 8B depicts the assay when a biological sample is applied (filled circle, 140) to the sample area of the LFA. The biological sample can contain antibodies (100) specific to a region of the isolated gE peptide (60) not linked to the solid support (130), but bound to a particle (90), in this case, via biotin (70) conjugated with the isolated gE peptide, and avidin (800 conjugated with the particle (90), which, collectively, is present in the conjugate region (150). As the biological sample migrates through the conjugate region (150), an antibody (100) peptide (60) conjugate is formed, where the antibody (100) is bound to an isolated gE peptide (60), which in turn is also bound, via biotin (70)/avidin (80) to the isolated gE peptide (60). This conjugate migrates further up to the test region (160). At the test region (160), the isolated gE peptide (60) linked to the solid support (130) binds the second arm of the antibody (100) resulting in the antibody (100) peptide (60) complex concentrating at the test line (160) to form a positive signal. As depicted, a control line (170) can also be included to ensure that the sample is migrating far enough to lead to a positive signal at the test line. The control line (170) turns color when a conjugate of a particle (90) and chicken IgY (120) binds to anti-chicken IgY (110) bound to the substrate (130).

FIG. 8C illustrates a portion of an example embodiment of a lateral flow assay as shown in FIGS. 8A and 813. FIG. 8C illustrates a component in the conjugate region including a particle (90), which is an example of a detectible agent, and is depicted as a circle attached to an antibody (120), such as chicken IgY, that is not specific to the isolated gE peptide (60) linked to the solid support (130) at the test region (160). Instead, the antibody (120) is specific to the control agent (110, depicted as a triangle), such as anti-chicken IgY, present in the control region (170) and bound to the substrate (130). In this manner, the conjugate region (150) can include a component that can be fused or separate from the isolated gE peptide (60). As the component migrates along the direction of flow, the component can reach the control region (170), where it comes into contact with the control agent (110) bound to the substrate (130) to form an antibody-target complex concentrating the detectible agent at the control region (170). Further, this signal can be detected independent of the presence of antibody (100) in the biological sample.

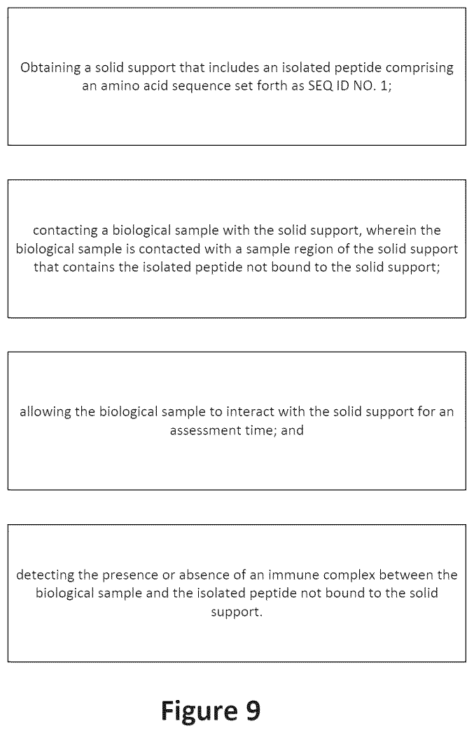

FIG. 9 illustrate a flow chart depicting an example method for using a LFA to detect the presence of an antibody in a biological sample. The method can include a sequence of steps that can be performed in a specified order or that may be rearranged. Further, the depicted method is provided for example only, different variations that include other steps or omit certain steps are also within the scope of the present disclosure. In the depicted embodiment, the method can include obtaining a solid support that includes an isolated peptide comprising an amino acid sequence set forth as SEQ ID NO. 1. The method can also include contacting a biological sample (e.g., blood and/or plasma) with the solid support, wherein the biological sample is contacted with a sample region of the solid support that contains (or is adjacent to a region containing) the isolated peptide not bound to the solid support. The method can also include allowing the biological sample to interact with the solid support for an assessment time. In general, aspects of example LFAs in accordance with the present disclosure provide for reduced assessment time compared to other assessment techniques such as ELISA. As such, for certain implementations the assessment time can be no less than 1 minute and no greater than 60 minutes. Finally, the method can include detecting the presence or absence of an immune complex between the biological sample and the isolated peptide not bound to the solid support. The immune complex, if present, may be formed between antibody present in the biological sample and the isolated peptide having the amino acid sequence set forth as SEQ ID NO. 1.

The present invention will be better understood with reference to the following non-limiting examples with reference to the foregoing drawings.

### EXAMPLES

The present examples provide aspects of embodiments of the present disclosure. These examples are not meant to limit embodiments solely to such examples herein, but rather to illustrate some possible implementations.

### Example 1: Representative Test Strips

### 1. VZV Membrane

CN95 membrane from Sartorius (or other suitable material) is striped with VZV gE (1-350aa) from Genscript, Zeba column exchanged into 1×PBS at 1 mg/mL. Control line is Donkey anti-Chicken at 0.5 mg/ml in 1×PBS.

The test line is striped 11 mm from the bottom edge of the membrane.

Control line is striped 16 mm from the bottom edge of the membrane.

### 2. VZV Conjugate

Streptavidin is conjugated to 0.4 nm Red Latex particles (or other suitable material) at a mass ratio of around 20:1 to around 40:1 (Beads:Protein)

VZV gE (1-350aa) from Genscript, Zeba column exchanged into 1×PBS was biotinylated with NHS-PEG12-Biotin at a molar ratio of around 25:1 (Biotin:Protein)

VZV gE-biotin was complexed with Streptavidin-Latex at a 2:1 molar ratio (VZV gE-biotin:Streptavidin-Latex)

Chicken IgY is conjugated to 0.4 nm Red Latex particles at a mass ratio of 40:1 (Beads:Protein)

Streptavidin-Latex/VZV gE-biotin conjugate complex was prepared in 50 mM Tris, 1% casein (7 day cured), 0.5% Tergitol, 10% sucrose, and 2% Trehalose to final concentration of 0.075% solids with Chicken IgY-Latex at a final concentration of 0.01% solids. These are merely representative concentrations of the various components, and other concentrations can be used.

### 3. VZV Conjugate Pad

An Ahlstrom 6614 glass fiber conjugate pad (or other suitable material) was cut to 1-20 mm. A conjugate solution with 0.075% Streptavidin-Latex/VZV gE-biotin conjugate complex was sprayed on to the 6614 polyester conjugate pad with 1 pass of 10 μL/cm towards the bottom of the pad.

### 4. VZV Sample Pad

GE MF1 glass fiber blood separator pad (or other suitable material) is cut to 1-20 mm.

### 5. Wick Pad

Ahlstrom 222 (or other suitable material) is cut to 1-40 mm and placed onto the backing card at the top with a 2 mm overlap with the membrane.

### 6. Final Test Strips

Final test strips are assembled with a 21 mm Ahlstrom 222 wicking pad placed flush with top edge of the 60 mm backing card and overlapping the top edge of the CN95 membrane by 2 mm. The 10 mm 6614 conjugate pad is placed overlapping the bottom edge of the CN95 membrane by 2 mm. An 10 mm MF1 Sample pad is placed overlapping the bottom edge of the 10 mm conjugate pad by 2 mm. Strips are cut to 4.9 mm wide, and placed in to the custom MICA 200 cassette.

### 7. Chase Buffer

In this example, the chase buffer is 12 mM Sodium Phosphate, 0.6M NaCl, and 1% Tergitol.

### 8. Test Procedure

Pipette approximately 10 μL of whole blood sample into the sample port.

Allow samples to fully absorb into the sample pad for 30 seconds.

Pipette 70 μL of chase buffer to the sample port.

Allow strips to run for 10 minutes before reading or interpreting results.

### 9. Layout

TABLE 1
VZV IgG Lateral Flow Assay Parameters

| | | | Location | |
|---|---|---|---|---|
| | | | from | |
| | | | bottom | |
| LFA Strip | Material | Length | of Strip | Thickness |
| Membrane | Sartorius | 1-40 mm | 1-25 mm | 100-800 μm |
| | CN95 | | | |
| Conjugate | Ahlstrom | 1-20 mm | 1-20 mm | 100-800 μm |
| Pad | 6614 | | | |
| Sample Pad | GE MF1 | 1-20 mm | 0 mm | 100-800 μm |
| Wick Pad | Ahlstrom | 1-40 mm | 1-80 mm | 100-2000 μm |
| | 222 | | | |
| Backing Card | Lohmann | 1-120 mm | N/A | 100-800 μm |

### 10. Test Principle

In one embodiment, the lateral flow test works as a sandwich assay, and is housed in a cassette. The test cassette comprises a test strip that includes a sample pad, conjugate pad, membrane, and wick pad.

The purpose of each component is as follows:
1) A patient sample is applied to a sample pad that contains a blood cell separator to facilitate flow of the sample fluid up the test strip;
2) A conjugate pad containing VZV gE recombinant antigen that is attached to Biotin and is complexed with Streptavidin conjugated to Red Colored Latex particles forming a VZV gE-Biotin: Streptavidin-Latex complex. Chicken IgY protein conjugated to Red Latex particles is also incorporated with the gE-Biotin:Streptavidin-Latex complex solution and is sprayed on towards the bottom of the conjugate pad and placed into a cassette;
3) A nitrocellulose membrane strip containing a striped donkey anti-Chicken IgG line (control line) and a striped VZV gE recombinant antigen line (test line); and
4) A wick pad that draws off liquid by capillary action.

When a correct volume of test blood, for example between 5 and 50 μl, more typically between 1- and 40 μl, and most typically, between 15 and 35 μl, sera or plasma specimen is dispensed into the sample pad of the test strip, the specimen migrates by capillary action along the test strip.

The anti-VZV IgG, if present in the specimen, will bind to the VZV gE-biotin:Streptavidin-Latex complex and further migrates to bind to the VZ gE antigen (test line) forming a red-colored test line, indicating a VZV IgG positive test result or no line will be visible to indicate a negative test result. The sample fluid will continue to run up the membrane over the control line. The chicken IgY-Latex complex will bind to anti-Chicken IgG protein on the control line and cause a visible red line to become visible regardless if the sample is positive or negative for VZV IgG. The results can be read within 10-20 minutes after the sample was added to the sample pad.

Using the test procedure (8) and the lateral flow assay layout (9), samples from different plasma donors were applied to LFA cassettes to demonstrate efficacy against a control donor having no antibodies.

### 11. ELISA and LFA Comparison—ELISA Method

Thaw serum and plasma samples.

Dilute each sample 21-fold in the sample diluent: 200 μL diluent+10 μL plasma/serum.

Vortex to mix.

Prepare the blank, negative, and positive controls from the VZV ELISA kit in the sample diluent at the same dilution as the samples.

200 μL diluent+10 μL controls

Vortex to mix.

Dilute the calibrator from 21-fold in the sample diluent:

400 μL diluent+20 μL calibrator

Vortex to mix.

Prepare 150 mL of wash buffer: 15 mL 10×Wash Buffer+135 mL water.

1. Add 100 μL of the prepared controls, calibrators, and to the VZV IgG ELISA plate.

Incubate plate for 25 min at room temperature with no shaking.

Dump liquid contents of plate into a 5 L pitcher.

Add 200 μL of wash buffer to each well.

Dump liquid contents of plate into a 5 L pitcher.

Repeat steps 4 and 5 for 5 total washes.

Add 100 μL of conjugate to each well in their respective plates

Incubate plate for 25 min at room temperature with no shaking.

Repeat steps 3-5 for 5 total washes.

Add 100 μL of TMB substrate to each well.

Protect the plates from light and allow them to develop for 12 min (official protocol is 10-15 min).

Add 50 μL of stop solution to each well.

Read plates at 450 nm using the Infinite F200PRO plate reader.

Analyze the results based on product instructions.

### 10. ELISA and LFA Comparison—LFA Method

TABLE 2
Materials and equipment.
Description

CN95 striped with Truncated VZV gE (1-350aa) PBS buffer exchanged @
1 mg/mL
Run Buffer - 12 mM Phosphate, 0.6M NaCl, 1% Tergitol
60 mm Backing Card, MIBA-020
Ahlstrom 222 wick pad 21 mm, 625074
8980 10 mm conjugate pad
MF1 10 mm sample pad
Sprayed conjugate complex @ 0.15%, 10 μL/cm onto 8980 pad without
treatment
6% Casein in 50 mM Tris
EDTA plasma from SLR
40% Sucrose in water
40% Trehalose in water
10% Tergitol
Chicken IgY-Latex 40:1

Laminate membrane on 1-120 mm backing card 1-30 mm from the bottom.

Laminate 222 (or other suitable material) wick pad flush to the top of the backing card and with a 1-10 mm overlap with the nitrocellulose membrane.

Laminate 1-20 mm 8980 conjugate pad (or other suitable material) with a 1-10 mm overlap with the nitrocellulose membranes.

Laminate MF1 1-20 mm blood separator pads flush to the backing card with a 1-10 mm overlap of the 6614 conjugate pad.

Cut into 1-20 mm strips.

Place in MICA 200 cassettes (or other suitable cassettes).

Add 1-50 μL of serum/plasma. Allow sample to absorb into the sample pad completely (~30 seconds).

Chase with 10-200 μL of buffer.

Allow the assay to run for 5-30 minutes.

Record the results.

Another embodiment of the test strip described herein is shown in the table below:

TABLE 3
Materials and equipment.
Materials and Equipment:
Description

FF120HP striped with gE (CHO - U0928FJ22001) @ 0.5 mg/mL rate: 0.5
μL/cm
8980 sprayed with SA-latex/gE-biotin conjugate complex (2:1) - Scale up
0.01″ thick 60 mm backing card, PN MIBA-020
Ahlstrom 222 wick pad 21 mm, 625074
MF1 10 mm sample pad
1X PBS
0.5M Sodium Phosphate
25% Tergitol
5M NaCl
Serum samples - Cross Reactivity
Chicken IgY-Latex 40:1

The test strip can be used in a manner analogous to that used with the test strip formulation shown in Table 2.

### Example 2: Representative Synthesis of the Isolated Peptides

In order to have isolated gE peptides, it is useful to have them expressed as recombinant peptides, for example, in CHO (Chinese hamster ovary) cells. Truncated VZV gE peptides were prepared using the following protocol:
Culture Volume: 1 L Expression
System: CHO-K1SP
Purification: Protein was obtained from supernatant, followed by AC,SEC and UFDF
Package: 43.00 ml. 40.00 ml/vial, 2 vials; 1.00 ml/vial, 3 vials
Concentration: 0.88 mg/ml
Purity: 96.58%, estimated by SEC-HPLC
Endotoxin Level: <3.00 EU/mg (LAL/TAL Endotoxin Assay)
Sterility: Sterilized via a 0.22 μm filter and packaged aseptically
Storage and Handling: Store at −80 °C. Aliquots should be stored at the same temperature after first use to avoid multiple freeze-thaws.
Storage Buffer: 20 mM NaAc—HAc, 150 mM NaCl pH 4.5

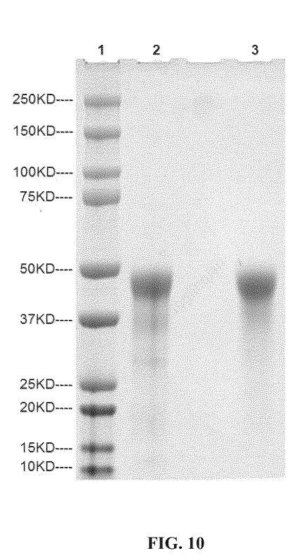

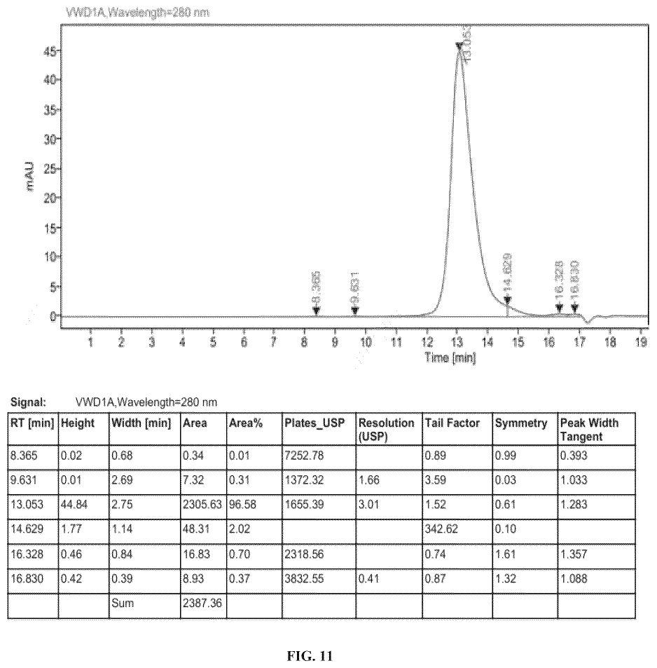

FIG. 10 is a slide resulting from gel electrophoresis (SDS-PAGE, or sodium dodecyl sulfate-polyacrylamide gel electrophoresis) of VZV TgE expression in CHO cells. Expected results are ~46 kDa for TgE. Lane 1: Protein Marker, (Bio-Rad Cat. No. 1610374); Lane 2: 5.00 μg truncated VZV gE under reducing conditions; Lane 3: 5.00 μg truncated VZV gE under non-reducing conditions. This proves that the isolated peptides used in the assays described herein can be prepared using conventional expression systems in conventional CHO cells, but other conventional methods of producing recombinant proteins, that also produce the isolated gE peptides, can also be used. FIG. 11 is an SEC-HPLC analysis of the truncated VZV gE, showing a purity of 96.58%. Accordingly, using this protocol, the isolated peptides described herein can be prepared.

All references referred to herein are hereby incorporated by reference for all purposes.

Many modifications and other embodiments of the invention will come to the mind of one skilled in the art having the benefit of the teachings presented in the foregoing descriptions and the associated drawings. Therefore, it is to be understood that the inventive concepts described herein are not to be limited to the specific embodiments disclosed, and that the modifications and embodiments are intended to be included within the scope of the dependent claims.

While certain exemplary embodiments of the inventive concept have been described and shown in the accompanying drawings, it is to be understood that such embodiments are merely illustrative of and not restrictive, and that the embodiments of the inventive concept not be limited to the specific constructions and arrangements shown and described, since various other modifications may occur to those ordinarily skilled in the art.

1. A solid support comprising at least two regions, each region defining an area of the solid support that includes an isolated peptide, wherein the isolated peptide has an amino acid sequence set forth as:
(SEQ ID 1)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHVHHHHHH,
or
(SEQ ID 2)
MGWSCIILLFLVATATGVHSSVLRYDDFHIDEDKLDTNSVYEPYYHSDHA
ESSWVNRGESSRKAYDHNSPYIWPRNDYDGFLENAHEHHGVYNQGRGIDS
GERLMQPTQMSAQEDLGDDTGIHVIPTLNGDDRHKIVNVDQRQYGDVFKG
DLNPKPQGQRLIEVSVEENHPFTLRAPIQRIYGVRYTETWSFLPSLTCTG
DAAPAIQHICLKHTTCFQDVVVDVDCAENTKEDQLAEISYRFQGKKEADQ
PWIVVNTSTLFDELELDPPEIEPGVLKVLRTEKQYLGVYIWNMRGSDGTS
TYATFLVTWKGDEKTRNPTPAVTPQPRGAEFHMWNYHSHV,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 1-2, and
d) functional groups for covalently attaching the isolated peptide sequences to a solid support.

2. The solid support of claim 1, wherein for one of the at least two regions, the isolated peptide is linked to the solid support.

3. The solid support of claim 1, wherein for one of the at least two regions, the isolated peptide is not linked to the solid support.

4. The solid support of claim 3, wherein the isolated peptide not linked to the solid support is fused to a heterologous protein, a detectable agent, a reactive agent, or combinations thereof.

5. The solid support of claim 4, wherein the detectable agent is a latex bead or a colloidal gold particle.

6. The solid support of claim 3, wherein the isolated peptide not linked to the solid support has a concentration of no less than 10 ng/mm² and no greater than 600 ng/mm² based on the area of the region.

7. The solid support of claim 2, wherein the isolated peptide linked to the solid support has a concentration of no less than 1 ng/mm² and no greater than 500 ng/mm² based on the area of the region.

8. The solid support of claim 1, wherein the solid support is not directly linked to an antibody.

9. The solid support of claim 1, wherein the solid support further comprises a control region, and wherein the control region comprises a control agent.

10. A solid support, wherein the support comprises:
a wicking material for directing material flow along a direction of the solid support;
a sample region comprising a first isolated peptide, wherein the first isolated peptide is not linked to the solid support, and wherein the first isolated peptide is fused to a heterologous protein; a detectable agent, a reactive agent, or combinations thereof;
a test region positioned beyond the sample region along the direction of material flow, wherein the test region comprises a second isolated peptide linked to the solid support; and
a control region positioned beyond the test region along the direction of material flow, wherein the control region comprises a control agent, and wherein each of the first isolated peptide and the second isolated peptide have an amino acid sequence set forth as:
(SEQ ID 1)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHVHHHHHH,
or
(SEQ ID 2)
MGWSCIILLFLVATATGVHSSVLRYDDFHIDEDKLDTNSVYEPYYHSDHA
ESSWVNRGESSRKAYDHNSPYIWPRNDYDGFLENAHEHHGVYNQGRGIDS
GERLMQPTQMSAQEDLGDDTGIHVIPTLNGDDRHKIVNVDQRQYGDVFKG
DLNPKPQGQRLIEVSVEENHPFTLRAPIQRIYGVRYTETWSFLPSLTCTG
DAAPAIQHICLKHTTCFQDVVVDVDCAENTKEDQLAEISYRFQGKKEADQ
PWIVVNTSTLFDELELDPPEIEPGVLKVLRTEKQYLGVYIWNMRGSDGTS
TYATFLVTWKGDEKTRNPTPAVTPQPRGAEFHMWNYHSHV,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 1-4, and
d) functional groups for covalently attaching the isolated peptide sequences to a solid support.

11. The solid support of claim 10, wherein the detectable agent is a latex bead or a colloidal gold particle.

12. The solid support of claim 10, wherein the first isolated peptide has a concentration of no less than 10 ng/mm2 and no greater than 600 ng/mm2 based on an area of the sample region.

13. A method for detecting anti-Varicella-Zoster Virus antibodies in a biological sample containing antibodies, comprising:
contacting the biological sample with a solid support including an isolated peptide, wherein the isolated peptide has an amino acid sequence set forth as:
(SEQ ID 1)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHVHHHHHH,
or
(SEQ ID 2)
MGWSCIILLFLVATATGVHSSVLRYDDFHIDEDKLDTNSVYEPYYHSDHA
ESSWVNRGESSRKAYDHNSPYIWPRNDYDGFLENAHEHHGVYNQGRGIDS
GERLMQPTQMSAQEDLGDDTGIHVIPTLNGDDRHKIVNVDQRQYGDVFKG
DLNPKPQGQRLIEVSVEENHPFTLRAPIQRIYGVRYTETWSFLPSLTCTG
DAAPAIQHICLKHTTCFQDVVVDVDCAENTKEDQLAEISYRFQGKKEADQ
PWIVVNTSTLFDELELDPPEIEPGVLKVLRTEKQYLGVYIWNMRGSDGTS
TYATFLVTWKGDEKTRNPTPAVTPQPRGAEFHMWNYHSHV,
and analogues thereof wherein the isolated peptide sequences comprise one or more of:
a) up to 5 amino acids at the C and/or at the N terminal ends,
b) one, two or three conservative amino acid substitutions, and
c) 98% or greater sequence homology to SEQ ID Nos. 1-2,
d) functional groups for covalently attaching the isolated peptide sequences to a solid support,
wherein contacting is performed under conditions sufficient to form an immune complex between the isolated peptide and an antibody present in the biological sample, and
detecting the presence or absence of the immune complex, wherein the presence of the immune complex indicates anti-VZV antibodies are present in the biological sample.

14. The method of claim 13, wherein a first portion of the isolated peptide is linked to the solid support.

15. The method of claim 14, wherein a second portion of the isolated peptide is not linked to the solid support.

16. The method of claim 15, wherein the isolated peptide not linked to the solid support is fused to a heterologous protein; a detectable agent, a reactive agent, or combinations thereof.

17. The method of claim 16, wherein the detectable agent is a latex bead, and wherein detecting the presence of or absence of the immune complex is determined based on presence of a color change on the solid support.

18. The method of claim 13, further comprising administering a therapeutic agent to a patient that tests positive for VZV infection, wherein the therapeutic agent is effective at treating the symptoms of a VZV infection, or in treating the underlying viral infection.

19. The method of claim 18, wherein the therapeutic agent is selected from the group consisting of acyclovir, valacyclovir, penciclovir, famciclovir, brivudin, foscarnet, vidarabine, interferon, and ribavirin.

20. The method of claim 13, further comprising administering a Shingles vaccine to the patient.

21. An isolated peptide having an amino acid sequence set forth as:
(SEQ ID 1)
MGWSCIILFLVATATGVHSSVLRYDDFHIDEDKLDTNSVYEPYYHSDHAE
SSWVNRGESSRKAYDHNSPYIWPRNDYDGFLENAHEHHGVYNQGRGIDSG
ERLMQPTQMSAQEDLGDDTGIHVIPTLNGDDRHKIVNVDQRQYGDVFKGD
LNPKPQGQRLIEVSVEENHPFTLRAPIQRIYGVRYTETWSFLPSLTCTGD
AAPAIQHICLKHTTCFQDVVVDVDCAENTKEDQLAEISYRFQGKKEADQP
WIVVNTSTLFDELELDPPEIEPGVLKVLRTEKQYLGVYIWNMRGSDGTST
YATFLVTWKGDEKTRNPTPAVTPQPRGAEFHMWNYHSHVHHHHHH,
or
(SEQ ID 2)
MGWSCIILLFLVATATGVHSSVLRYDDFHIDEDKLDTNSVYEPYYHSDHA
ESSWVNRGESSRKAYDHNSPYIWPRNDYDGFLENAHEHHGVYNQGRGIDS
GERLMQPTQMSAQEDLGDDTGIHVIPTLNGDDRHKIVNVDQRQYGDVFKG
DLNPKPQGQRLIEVSVEENHPFTLRAPIQRIYGVRYTETWSFLPSLTCTG
DAAPAIQHICLKHTTCFQDVVVDVDCAENTKEDQLAEISYRFQGKKEADQ
PWIVVNTSTLFDELELDPPEIEPGVLKVLRTEKQYLGVYIWNMRGSDGTS
TYATFLVTWKGDEKTRNPTPAVTPQPRGAEFHMWNYHSHV,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 1-2,
d) one or more functional groups for covalently attaching the isolated peptide sequences to a solid support, and
e) a solid support to which the isolated peptide is attached, through the functional group or
f) a particle to which the isolated peptide is attached, either covalently or non-covalently.

22. The isolated peptide of claim 21, further comprising a VZV antibody bound to the isolated peptide bound to the particle.

23. The isolated peptide of claim 22, wherein the VZV antibody is bound to a) an isolated peptide bound to a particle, and b) an isolated peptide bound to a solid support.
